(19) United States
(12) Patent Application Publication
(10) Pub. No.: US 2023/0139125 A1
(43) Pub. Date: May 4, 2023

(54) DIETARY SUPPLEMENT FOR IMPROVING BRAIN PERFORMANCE

(71) Applicant: Richard Natrillo, Boca Raton, FL (US)

(72) Inventor: Richard Natrillo, Boca Raton, FL (US)

(21) Appl. No.: 17/912,371

(22) PCT Filed: Feb. 3, 2022
(86) PCT No.: PCT/US2022/015114

§ 371 Date: Sep. 16, 2022

Related U.S. Application Data
(60) Provisional application No. 63/145,537, filed on Feb. 4, 2021.

(51) Int. Cl. A23L 33/16; A23L 33/15; A23L 33/105; A23L 33/115; A61K 31/4415; A61K 31/714; A61K 33/10; A61K 31/685; A61K 31/6615; A61K 9/00

(52) U.S. Cl. CPC A23L 33/16; A23L 33/15; A23L 33/105; A23L 33/115; A61K 9/0095; A61K 31/685; A61K 31/714; A61K 31/4415; A61K 31/6615; A61K 33/10

(57) ABSTRACT

Disclosed herein is a dietary supplement containing alpha-glyceryl phosphoryl choline, phosphatidylserine, and an alkalizing agent, and its use in methods of maintaining or improving brain functions, such as mental focus, clarity, long-term memory, short-term memory, analytical reasoning, higher learning, mental processing, mental clarity, creativity, procrastination, willpower, reasoning, mood, retrieval/recall, learning gradient, and reaction time. The supplement can also be used in methods of treating or preventing cognitive dysfunction and dementia.

### CROSS REFERENCE TO RELATED APPLICATIONS

The present patent application claims priority benefit to U.S. Provisional Application No. 63/145,537 filed on Feb. 4, 2021, the entire content of which is incorporated herein by reference. All references cited anywhere in this specification, including the background and detailed description sections, are incorporated by reference as if each had been individually incorporated.

### FIELD OF THE INVENTION

The present invention relates generally to a dietary supplement for improving brain performance comprising naturally occurring ingredients. There is a rising demand for multi-utility drugs that work as antidepressants, antianxiety, and energy/metabolism boosters. A growing number of students, e-gamers, professionals, athletes, and the elderly have turned to so-called “smart drugs” to improve their attention and focus. However, many of these drugs contain undesirable synthetic components, making them unattractive to the growing global vegan population, as well as the 50% of American consumers who prefer products with natural ingredients. What is needed is a natural dietary supplement for improving brain performance. The present invention accomplishes this via alpha-glyceryl phosphoryl choline and phosphatidylserine in combination with an alkalizing agent, and is used to improve a variety of cognitive functions.

### BACKGROUND

Nootropics, also known as “smart drugs”, are one of the most popular supplement categories currently on the market, focusing on brain and memory health. They are used to improve memory, focus, mental energy, and problem-solving skills. Their wide-spread demand is attributed to the 73 million baby boomers living in America, as well as college students and career-driven individuals striving for excellence. Hundreds of different formulations have flooded the market in recent years, all promising unrealistic benefits due to insufficient doses, ineffective ingredients, and low-quality fillers. A good nootropic helps to improve memory, promote focus and alertness, enhance learning, support verbal recall and analytical thinking, boost mental energy, enhance creativity, reduce stress, and/or fight cognitive decline. There is a continuing need for natural nootropics that provide a wide range of the desired benefits.

### SUMMARY OF THE INVENTION

The invention includes a dietary supplement for the improvement of brain performance. One general aspect of the invention discloses a dietary supplement including alpha-glyceryl phosphoryl choline, phosphatidyl serine, and an alkalizing agent. The dietary supplement may also include one or more of bacopa or bacopa extract, L-theanine, beet root extract, ashwagandha, and acetyl-L-camitine. The dietary supplement may further include one or more of coffee fruit concentrate, Lion’s mane mushroom, Rhodiola rosea root extract, and huperzine-A. In certain embodiments, the caffeine is from Coffea robusta and the lion’s mane mushroom is organic lion’s mane mushroom. In certain embodiments, the huperzine-A is derived from Huperzia serrata whole herb extract. In certain embodiments, the beet root extract is oxygen enriched. In certain embodiments, the Rhodiola rosea root extract may include 3% rosavins and 1% salidrosides. In certain embodiments, the ashwagandha root extract may include 2.5% withanolides. In certain embodiments, the coffee fruit concentrate is coffee berry whole fruit powder.

In one aspect, the alkalizing agent may include one or more carbonate salts, one or more bicarbonate salts, or a combination thereof. The one or more bicarbonate salts may be sodium bicarbonate, potassium bicarbonate, or a combination thereof, and said one or more carbonate salts may be potassium carbonate, magnesium carbonate, or a combination thereof.

In another aspect, the dietary supplement may include vitamin B6 and vitamin B12, and further may include citric acid, natural flavors, silica, and highly refined steviol glycosides. In certain embodiments, the supplement is formulated as a fine powder.

In another aspect, the invention discloses a method of improving brain function by orally administering an effective amount of the dietary supplement. Brain function may include improving at least one of mental focus, clarity, long-term memory, short-term memory, analytical reasoning, higher learning, mental processing, mental clarity, creativity, procrastination, willpower, reasoning, mood, retrieval/recall, learning gradient, and reaction time. In embodiments, a daily dose of the dietary supplement has a total mass of about 9.5 g and is added to about 8-10 ounces (235-300 mL) of a liquid, preferably water, prior to administration. After dissolving or suspending in liquid, the dietary supplement can be administered by drinking.

In another aspect, the invention discloses a method of treating or preventing cognitive dysfunction in subject by orally administering an effective amount of the dietary supplement.

In another aspect, the invention discloses a method of treating or preventing dementia in a subject by orally administering an effective amount of the dietary supplement.

Further objectives and advantages, as well as the structure and function of preferred embodiments will become apparent from a consideration of the description, and examples.

### DETAILED DESCRIPTION OF THE INVENTION

Embodiments of the invention are discussed in detail below. In describing embodiments, specific terminology is employed for the sake of clarity. However, the invention is not intended to be limited to the specific terminology so selected. While specific exemplary embodiments are discussed, it should be understood that this is done for illustration purposes only. A person skilled in the relevant art will recognize that other components and configurations can be used without parting from the spirit and scope of the invention. All references cited herein are incorporated by reference as if each had been individually incorporated.

In the description and examples that follow, unless otherwise indicated, all parts and percentages are by weight.

As used herein, the term “about” refers to plus or minus 10% of the indicated value. Unless otherwise stated or made clear by context, weight percentages are provided based on the total amount of the composition in which they are described.

As used herein, the singular forms “a,” “an,” and “the” include plural referents unless the context clearly dictates otherwise.

Alpha-glyceryl phosphoryl choline and phosphatidylserine have shown promising results as nootropics. Alpha-glyceryl phosphoryl choline (or Alpha-GPC) is a cholinergic compound with neuroprotective activity that prevents cognitive decline while simultaneously increasing attention, improving mood, and boosting mental energy. It is especially prized for its ability to improve memory, and its high bioavailability makes it a great source of choline for producing the neurotransmitter acetylcholine, which can lead to increased alertness and clarity of thought. In conjunction, Alpha-GPC boosts the development of new brain cells and enhances the brain’s ability to repair damaged cellular membranes.

Phosphatidylserine (or PS) is a phospholipid component of the membrane encasing brain cells to help keep them fluid and permeable. PS is integral to cleaning up damaged neurons by promoting neuron repair and maintenance. It also promotes nerve growth factor (NGF) for neurogenesis and neuroplasticity. This neuroplasticity helps neurons form new connections needed for memory formation. PS can also boost mental energy by easing the flow of glucose and oxygen needed to power brain cells, thereby maintaining an optimized brain. It can work in concert with Omega-3 fatty acids such as DHA to keep brain cells optimized to prolong neuron survival and health.

While Alpha-GPC and PS are present in a variety of nootropics on the market, the present invention overcomes the short-comings of existing supplements by combining the full clinically efficacious doses of both these ingredients with an alkalizing agent, as well as other additional ingredients with complimentary nootropic properties.

The present invention also provides a wide range of beneficial effects including immediate effects upon administration and long-term effects obtained from administration over an extended time period. Immediate benefits can be realized in as little as 30 minutes, although reaction times will vary by individual. Continued administration helps to maintain and improve memory and cognition. The present invention is useful in supporting and maintaining brain health and function.

### Components

### Alpha-Glyceryl Phosphoryl Choline (Alpha-GPC)

Alpha-GPC has been extensively studied in human clinical research and is generally regarded as safe (GRAS) by the FDA. It is a tasteless, water soluble, and stable acetylcholine and phosphatidylcholine precursor with widespread use across a variety of health-related applications, including autism, cognition, growth hormones, exercise performance, and sleep.

In the brain, acetylcholine plays an important role in cognitive function, including arousal, attention, memory, and motivation. Disruptions in acetylcholine functions are correlated with memory problems prevalent in Alzheimer’s Disease (i.e. AD), dementia, and other neurocognitive disorders. The acetylcholine delivered by Alpha-GPC helps to target optimal acetylcholine levels to maintain and improve memory, concentration, focus, recall of names and numbers, mental energy, and learning. These improved mental health performance outcomes are also observed during and after stressful exercise as boosts in power, strength, reaction time, and agility. Alpha-GPC is also a precursor to membrane phospholipids, and therefore could potentially improve neuronal membrane fluidity.

Other benefits of optimal acetylcholine levels are healthy metabolism, liver function, lipid clearance, cholesterol health, blood pressure, and homocysteine levels.

In an embodiment of the invention, the Alpha-GPC is provided as either Alpha-GPC, or as a formulation containing active Alpha-GPC together with excipients and/or fillers. For example, the Alpha-GPC can be provided as Alphasize™ (ChemiNutra, Austin, TX) which contains about 50% of active Alpha-GPC.

In an embodiment of the invention, the dietary supplement is formulated so that a daily dose provides about 200 to about 400 mg of active Alpha-GPC. In an embodiment of the invention, the dietary supplement is formulated so that a daily dose provides about 200 mg, about 250 mg, about 300 mg, about 350 mg, or about 400 mg of active Alpha-GPC. In a preferred embodiment, the dietary supplement is formulated so that a daily dose provides 300 mg of active Alpha-GPC.

### Phosphatidylserine (PS)

PS has a wide variety of scientifically substantiated health benefits, including improving memory, learning, recall, and focus, reducing stress, enhancing exercise performance and reducing muscle soreness, and improving healthy aging. It is a natural phospholipid compound produced in the body, but nutritional deficiencies in the typical diet prevents the body from producing sufficient levels of PS to optimally support brain and memory functions. These insufficient natural levels continue to decrease with age, making PS supplementation critical to replenish cognitive functions of the brain.

Supplementation with PS has been shown to aid neurons in the brain and support all cognitive tasking including memory, recall, focus, as well as anti-stress benefits. In a 2010 study investigating the ability of PS to slow age-related memory loss, it was found that at the end of a six-month period participants with relatively low memory scores at the start of the study experienced a significant improvement in memory when compared to placebo. It has also been shown to be beneficial to aid in age-related cognitive decline and dementia in the elderly.

While a vast majority of the studies have focused on its benefits in age-related cognitive decline, more recent studies have suggested PS can also be a beneficial dietary supplement to address memory loss associated with alcohol consumption as well as over the counter drugs.

In an embodiment of the invention, the PS is provided as either PS, or a PS formulation that contains active PS together with excipients and/or fillers. For example, the PS can be provided as SerinAid™ (ChemiNutra, Austin, TX) which contains about 50% of active PS.

In an embodiment of the invention, the dietary supplement is formulated so that a daily dose provides about 200 to about 400 mg of active PS. In an embodiment of the invention, the dietary supplement is formulated so that a daily dose provides about 200 mg, about 250 mg, about 300 mg, about 350 mg, or about 400 mg of active PS. In a preferred embodiment, the dietary supplement is formulated so that a daily dose provides 300 mg of active PS.

In a preferred embodiment of the invention, the weight ratio of active PS to active Alpha-GPC is about 1:1.

### Alkalizing Agent

Modem day lifestyles are prone to poor diet, stressful daily routine, smoking, and pollution, all of which create more acid in the blood and destroy the body’s necessary alkaline environment. To address these issues, the nootropic dietary supplement of the invention includes an alkalizing agent. When the dietary supplement of the invention is added to water for administration or consumption, the alkalizing agent provides the benefits of an alkaline water.

The alkalizing agent increases energy levels, hydration, and mental alertness to combat fatigue and promote peak bodily performance. By flushing out acidic waste and toxins, the alkalizing agent supports wellness via detox and weight loss. It can also improve nutrient absorption by eliminating mucus buildup on colon walls, allowing for improved delivery of supplements and nutrients. The additive will also neutralize free radicals and acids in the bloodstream to reduce inflammation, and reduce buildup of lactic acid in the muscles to enhance athletic endurance.

When the body lacks bicarbonate, an acidic environment is created wherein the blood is incapable of neutralizing poisonous acids, including uric acid, sulfuric acid, and phosphoric acid. This acidic environment promotes the formation of cancer cells as they are acidic in nature whereas healthy cells are alkaline. The body responds by taking calcium from the bones to form urate, phosphate, and sulfate salts, resulting in bone loss. Accordingly, the alkalizing agent can promote stronger bone formation by eliminating this calcium robbing.

Sufficient bicarbonate is also necessary to lower the risk of blood clots and debris accumulation in the bloodstream. Therefore, bicarbonate is necessary to reduce the risk of heart attack and stroke. Other uses include immune system support, deceleration of the aging process, and aiding in the body’s adaptation to changes in altitude.

In an embodiment of the invention, the alkalizing agent is composed of bicarbonate and carbonate. While any source of bicarbonate and carbonate is suitable for use in the invention, in a preferred embodiment the bicarbonate and carbonate are provided as salts. These salts can include both organic and inorganic salts. In a preferred embodiment the salts are inorganic salts. In an exemplary embodiment the inorganic salts are selected from one or more of sodium, potassium, and/or magnesium salts. In an embodiment the sodium salt is sodium bicarbonate, the potassium salt is potassium bicarbonate and/or potassium carbonate, and the magnesium salt is magnesium carbonate.

In an embodiment of the invention, the dietary supplement contains a sufficient amount of the alkalizing agent such that, upon consumption, the supplement constructs an alkaline environment. The dietary supplement contains sufficient alkalizing agent to provide an alkaline drink when mixed with water for consumption having a pH that ranges from about 8 to about 11. In embodiments, the alkalizing agent constructs an alkaline environment that has a pH of about 8.0, about 8.5, about 9.0, about 9.5, about 10.0, about 10.5, or about 11.0. In an embodiment of the invention, the dietary supplement contains about 5% to about 15% by weight of the alkalizing agent. In an embodiment of the invention, the dietary supplement contains about 5%, about 6%, about 7%, about 8%, about 9%, about 10%, about 11%, about 12%, about 13%, about 14%, or about 15% of the alkalizing agent. In a preferred embodiment, the dietary supplement contains about 10% of the alkalizing agent.

In an embodiment of the invention, the dietary supplement is formulated so that the alkalizing agent provides a daily dose of about 800 to about 1200 mg of carbonate and/or bicarbonate, from the alkalizing agent. In an embodiment of the invention, the dietary supplement is formulated so that the alkalizing agent provides a daily dose of about 800 mg, about 820 mg, about 840 mg, about 860 mg, about 880 mg, about 900 mg, about 920 mg, about 940 mg, about 960 mg, about 980 mg, about 1000 mg, about 1020 mg, about 1040 mg, about 1060 mg, about 1080 mg, about 1100 mg, about 1120 mg, about 1140 mg, about 1160 mg, about 1180 mg, or about 1200 mg of carbonate and/or bicarbonate. In a preferred embodiment, the dietary supplement is formulated so that the alkalizing agent provides a daily dose of about 920 mg of carbonate and/or bicarbonate.

In an embodiment of the invention, the dietary supplement is formulated so that the alkalizing agent provides a daily dose of about 90 mg to about 120 mg of sodium, present as sodium bicarbonate, from the alkalizing agent. In an embodiment of the invention, the dietary supplement is formulated so that the alkalizing agent provides a daily dose of about 90 mg, about 95 mg, about 100 mg, about 105 mg, about 110 mg, about 115 mg, or about 120 mg of sodium as sodium bicarbonate. In a preferred embodiment, the dietary supplement is formulated so that the alkalizing agent provides a daily dose of about 105 mg of sodium present as sodium bicarbonate. For example, when the dietary supplement provides about 384 mg of sodium bicarbonate, about 105 mg sodium is present.

In an embodiment of the invention, the dietary supplement is formulated so that the alkalizing agent provides a daily dose of provides about 380 mg to about 480 mg of potassium present as potassium carbonate and/or potassium bicarbonate, from the alkalizing agent. In an embodiment of the invention, the dietary supplement is formulated so that the alkalizing agent provides a daily dose of about 380 mg, about 390 mg, about 400 mg, about 410 mg, about 420 mg, about 430 mg, about 440 mg, about 450 mg, about 460 mg, about 470 mg, or about 480 mg of potassium present as potassium carbonate and/or potassium bicarbonate. In a preferred embodiment, the dietary supplement is formulated so that the alkalizing agent provides a daily dose of about 430 mg of potassium present as potassium carbonate and/or potassium bicarbonate. In an exemplary preferred embodiment, the dietary supplement is formulated so that the alkalizing agent provides a daily dose of about 600 mg of potassium bicarbonate and about 350 mg of potassium carbonate, or about 234 mg of potassium as potassium bicarbonate; and about 196 mg of potassium as potassium carbonate, for a total of about 430 mg of potassium.

In an embodiment of the invention, the dietary supplement is formulated so that the alkalizing agent provides a daily dose of about 40 mg to about 60 mg of magnesium present as magnesium carbonate. In an embodiment of the invention, the dietary supplement is formulated so that the alkalizing agent provides a daily dose of about 40 mg, about 45 mg, about 50 mg, about 55 mg, or about 60 mg of magnesium present as magnesium carbonate. In a preferred embodiment, the dietary supplement is formulated so that the alkalizing agent provides a daily dose of about 50 mg of magnesium present as magnesium carbonate. For example, when the dietary supplement contains about 173 mg of magnesium carbonate, about 50 mg of magnesium is present.

According to an embodiment of the invention, the dietary supplement contains active Alpha-GPC active PS, and an alkalizing agent in amounts effective for the alkalizing agent to provide synergistic absorption and activity effects of the active Alpha-GPC and active PS. Embodiments of the invention can further include one or more of bacopa (or bacopa extract), L-theanine, beet root extract, ashwaghanda, acetyl-L-carnitine, coffee fruit extract, Lion’s mane mushroom, Rhodiola rosea root extract, huperzine-A, and caffeine (for example from Coffea robusta). As described further below, the additional ingredients can contribute to the overall nootropic effect of the invention, and can also provide improvements in immediate and long-term effects provided by administration of the dietary supplement of the invention.

### Whole Coffee Fruit Concentrates

Clinical research throughout the years has led to the discovery that ingesting small amounts of whole coffee fruit concentrates, which may be, for example, coffee berry extracts, and coffee berry whole fruit powders, significantly increases brain-derived neurotrophic factor (i.e. BDNF), a protein crucial to maintaining healthy neurons and creating new ones. The effects are not obtained from green coffee beans, chlorogenic acids, or drinking coffee. It is only the whole coffee fruit products that increase BDNF to amounts sufficient to naturally support declining neuroprotection levels in humans. This has led to the development of products such as NeuroFactor® (FutureCeuticals, Momence, IL), an all-natural, caffeine free, product made from the whole fruit, bean included, of the coffee plant Coffea arabica.

Coffee fruit concentrates have also been shown to provide nutrients required to help the aging brain decrease oxidative stress, which can help to balance the brain and help improve memory and learning.

In a preferred embodiment of the invention, the coffee fruit concentrate is provided as a coffee berry whole fruit powder.

In an embodiment of the invention, the dietary supplement is formulated so that a daily dose provides about 50 mg to about 150 mg of coffee fruit concentrate. In an embodiment of the invention, the dietary supplement is formulated so that a daily dose provides about 50 mg, about 60 mg, about 70 mg, about 80 mg, about 90 mg, about 100 mg, about 110 mg, about 120 mg, about 130 mg, about 140 mg, or about 150 mg of coffee fruit concentrate. In a preferred embodiment, the dietary supplement is formulated so that a daily dose provides about 100 mg of coffee fruit concentrate. In a most preferred embodiment, the dietary supplement is formulated so that a daily dose provides about 100 mg of coffee berry whole fruit powder.

### Acetyl-L-Carnitine (ALC)

Acetyl-L-Carnitine (or ALC) is a mitochondriotropic substance necessary for optimal mitochondrial function that readily crosses the blood-brain barrier (i.e. BBB). It acts as defense for mitochondrial metabolism and provides structural integrity to the mitochondrial and plasma membranes upon which cell survival and neurotransmission depend.

ALC has multiple functions, including facilitation of mitochondrial energy supply and enhancement of membrane stability and cholinergic transmission. Its neuroprotective activity makes ALC a readily available substrate that can spark mitochondrial energy-linked processes, playing an important role in the synthesis and maintenance of cell membranes to optimize membrane fluidity.

Maintaining optimal ALC levels helps protect various systems directly via an increase in metabolic potential and indirectly via improvement of the secondary line antioxidant defense system. This system prevents or reduces the risk of chronic diseases linked to cognitive decline. This unique activity also directly augments the function of the central nervous system (CNS), resulting in increased cognitive potential.

ALC has demonstrated positive actions on several cerebral functions, including mental performance, learning, concentration, and mood levels. This has resulted in its clinical indications in Alzheimer’s, dementia, cerebral ischemia and reperfusion, attention deficit hyperactivity disorder (i.e. ADHD), depression/mood, peripheral/diabetic neuropathy, and antiretroviral neuropathy commonly associated with HIV medication.

In an embodiment of the invention, the dietary supplement is formulated so that a daily dose provides about 500 mg to about 1500 mg of ALC. In an embodiment of the invention, the dietary supplement is formulated so that a daily dose provides about 500 mg, about 600 mg, about 700 mg, about 800 mg, about 900 mg, about 1000 mg

, about 1100 mg, about 1200 mg, about 1300 mg, about 1400 mg, or about 1500 mg of ALC. In a preferred embodiment, the dietary supplement is formulated so that a daily dose provides about 1000 mg of ALC.

### Huperzine-A (HupA)

Huperzine-A (or HupA) is isolated from Huperzia serrata whole herb extract, which is used in traditional Chinese Medicine. HupA is an alkaloid capable of crossing the BBB with cognitive enhancing and neuroprotective properties, and has been suggested for the treatment of AD. HupA’s cognitive-enhancing effects are attributed to its potent, reversable acetylcholinesterase inhibition properties that have exhibited memory enhancing efficacy in both dementia and AD.

Studies on the non-cholinergic roles of HupA have made important contributions to the understanding of the pharmacological mechanism of HupA in the treatment of AD. These non-cholinergic properties include the ability to protect neurons against Ab-induced oxidative injury and apoptosis, ameliorate mitochondrial malfunction, antagonize NMDA-R, regulate NGF, promote non-amyloidogenic APP processes, and reduce iron in the brain. This multiplicity of action not only renders HupA a highly effective drug with the potential to serve as a disease-modifying agent for AD, but also suggests HupA could prove useful in the treatment of other neurological disorders.

In an embodiment of the invention, the HupA is provided as HupA from Huperzia serrata whole herb extract.

In an embodiment of the invention, the dietary supplement is formulated so that a daily dose provides about 100 µg to about 300 µg of HupA. In an embodiment of the invention, the dietary supplement is formulated so that a daily dose provides about 100 µg, about 150 µg, about 200 µg, about 250 µg, or about 300 µg of HupA. In a preferred embodiment, the dietary supplement is formulated so that a daily dose provides about 200 µg of HupA.

### L-Theanine and Caffeine

L-Theanine is an amino acid found in tea and some species of mushrooms. It is similar in structure to glutamic acid, which is another amino acid that acts as an excitatory amino acid neurotransmitter. Some people take theanine orally for anxiety and stress, depression, schizophrenia, and for the prevention of AD. It also has implications in the treatment of ADHD, with an ability to improve attention in such subjects.

In terms of mental performance, theanine has been shown to help reduce errors when taken before tests, and its combination with caffeine has been shown to improve one’s ability to switch attention between different tasks.

Owen et al. (Nutritional Neuroscience, 2008, Vol. 11, Issue No. 4, 193-198) found that the combination of 100 mg of L-theanine with 50 mg of caffeine improved subject alertness, and speed and accuracy of attention-switching tasks. There were also demonstrated reductions in susceptibility to distracting information in memory tasks. The results replicated previous evidence suggesting that L-theanine and caffeine in combination are beneficial for improving performance of cognitively demanding tasks.

Other biomedical benefits of theanine include reducing blood pressure, preventing the flu, and improving how well cancer drugs work as well as decreasing their side effects.

In an embodiment of the invention, the dietary supplement is formulated so that a daily dose provides about 150 mg to about 250 mg of L-theanine. In an embodiment of the invention, the dietary supplement is formulated so that a daily dose provides about 150 mg, about 200 mg, or about 250 mg of L-theanine. In a preferred embodiment, the dietary supplement is formulated so that a daily dose provides about 200 mg of L-theanine.

Caffeine is a central nervous system simulant. It is member of the methylxanthine class of compounds, and the most widely consumed psychoactive drug in the world. While several mechanisms of action are known, the most prominent is its ability to reversibly block the action of adenosine on its receptors, thereby preventing the onset of drowsiness. For most healthy adults, up to 400 mg of caffeine a day is widely regarded as safe.

In an embodiment of the invention, the dietary supplement contains caffeine derived from a natural source, such as coffee or tea. In a preferred embodiment, the dietary supplement of the invention contains caffeine from Coffea robusta.

In an embodiment of the invention, the dietary supplement is formulated so that a daily dose provides about 20 mg to about 80 mg of caffeine. In an embodiment of the invention, the dietary supplement is formulated so that a daily dose provides about 20 mg, about 30 mg, about 40 mg, about 50 mg, about 60 mg, about 70 mg, or about 80 mg of caffeine. In a preferred embodiment, the dietary supplement contains about 50 mg of caffeine. In a most preferred embodiment, the dietary supplement is formulated so that a daily dose provides about 50 mg of caffeine from Coffea robusta.

In exemplary embodiments, the dietary supplement of the invention contains both L-theanine and caffeine.

### Beet Root Extract

Beet root extract contains nitrates which can help increase blood flow to the brain, thereby improving cognitive function and potentially reducing the risk of dementia. This increased blood flow also helps carry oxygen to the brain, thereby aiding in neurovascular coupling, an intricate method by which harder working brain cells “ask” for and receive more oxygen. This process involves a complex array of cells and signaling molecules that work to i) boost blood flow to the localized brain cells that need a temporary increase in oxygen to operate at maximum efficiency, ii) carry oxygen to the brain, iii) keeps blood pressure in check, and iv) improves athletic performance.

Other health implications of beet root extract include fighting inflammation, improving digestive health, weight loss, as well as some anti-cancer properties.

In an embodiment of the invention, the dietary supplement is formulated so that a daily dose provides about 500 mg to about 1500 mg of beet root extract. In an embodiment of the invention, the dietary supplement is formulated so that a daily dose provides about 500 mg, about 600 mg, about 700 mg, about 800 mg, about 900 mg, about 1000 mg, about 1100 mg, about 1200 mg, about 1300 mg, about 1400 mg, or about 1500 mg of beet root extract. In a preferred embodiment, the dietary supplement is formulated so that a daily dose provides about 1000 mg of beet root extract.

In a preferred embodiment of the invention, the beet root extract is an oxygen enriched beet root extract.

### Mushrooms/Lion’s Mane Mushrooms

The culinary mushroom most extensively studied for its neurohealth properties is Hericium erinaceus, also known as lion’s mane mushroom. Polysaccharides present in aqueous extracts of Hericium erinaceus can potentially induce neuronal differentiation and promote neuronal survival. These studies suggest that the mushroom can be consumed either fresh or processed. Two other components present in the extracts, hericonones and erinacines, have been shown to stimulate the growth of brain cells.

Lion’s mane mushroom extracts have proven efficacy in the reduction of symptoms of memory loss in mice, as well as the prevention of neuronal damage caused by amyloid-beta plaques, the major contributing factor in the development, onset, and progression of AD. A study in older adults with mild cognitive impairment found that consuming three grams of powdered lion’s mane mushroom daily for four months significantly improved mental functioning. These improvements quickly faded when supplementation ceased.

These results suggest that lion’s mane mushroom contains compounds that stimulate brain cells and protect them from damage caused by AD.

Mushroom components can be provided in several forms, for example as an extract, as a whole mushroom, or as parts of a mushroom, which or dried or processed. Other mushroom species with neurite outgrowth and neuronal health benefits include Ganoderma lucidum, Grifola frondosa, and Sarcodon scabrosus. In exemplary embodiments, the mushroom component is mycelium and fruiting body components of organic lion’s mane mushroom.

In an embodiment of the invention, the dietary supplement contains a mushroom with neuronal health benefits selected from lion’s mane mushroom, Ganoderma lucidum, Grifola frondosa, or Sarcodon scabrosus. In an embodiment of the invention, the mushroom is an organically harvested mushroom. In a preferred embodiment the mushroom is lion’s mane mushroom. In another preferred embodiment, the lion’s mane mushroom is an organic lion’s mane mushroom.

In an embodiment of the invention, the dietary supplement is formulated so that a daily dose provides about 150 mg to about 250 mg of mushroom. In an embodiment of the invention, the dietary supplement is formulated so that a daily dose provides about 150 mg, about 200 mg, or about 250 mg of mushroom. In a preferred embodiment, the dietary supplement is formulated so that a daily dose provides 200 mg of mushroom. In a most preferred embodiment, the dietary supplement is formulated so that a daily dose provides about 200 mg of lion’s mane mushroom.

### Bacopa

Bacopa, from Bacopa monnieri leaf extract, has demonstrated and significant cognitive enhancing benefits when administered chronically. A 2004 double-blind, placebo-controlled study found that healthy individuals administered 300 mg of bacopa extract that contained 55% combined bacosides over the course of 12 weeks had a significant improvement in verbal learning, memory consolidation, and speed of early information processing in the treatment group compared to placebo. Studies also suggest that bacopa extract may improve some measures of memory and hand-eye coordination in children aged 6-8 years. However, results have been inconsistent across studies, suggesting that some bacopa extracts are efficacious in improving memory, while others are not.

To address these inconsistencies, a randomized, double-blind, placebo-controlled 2008 study was conducted using a whole plant standardized Bacopa monnieri extract to assess cognitive performance, anxiety, and depression in the elderly. The safety and tolerability of the standardized extract was also investigated. The bacopa participants had enhanced AVLT delayed word recall memory scores relative to the placebo group. Stroop results were also significant, with improvements in the bacopa group observed while the placebo group results remained unchanged. CESD-10 depression scores, combined state plus train anxiety scores, and heart rate all decreased over time in the bacopa group, but increased for the placebo group. No effects were found on the DAT, WAIS digit task, mood, or blood pressure, suggesting the dose was well tolerated, and little adverse side effects were observed. The study provided further evidence that bacopa can safely and effectively enhance cognitive performance in the aging population.

In an embodiment of the invention, the bacopa is provided as bacopa or a bacopa extract, for example a whole plant extract.

In an embodiment of the invention, the dietary supplement is formulated so that a daily dose provides about 200 mg to about 400 mg of bacopa or bacopa extract. In an embodiment of the invention, the dietary supplement is formulated so that a daily dose provides about 200 mg, about 250 mg, about 300 mg, about 350 mg, or about 400 mg of bacopa or bacopa extract. In a preferred embodiment, the dietary supplement is formulated so that a daily dose provides 300 mg of bacopa or bacopa extract.

### Rhodiola Rosea Root Extract

Rhodiola rosea root extract may help protect cells from damage, regulate heartbeat, and has the potential for improving learning and memory.

In an embodiment of the invention, the Rhodiola rosea root extract contains 3% rosavins and 1% salidrosides.

In an embodiment of the invention, the dietary supplement is formulated so that a daily dose provides about 50 mg to about 250 mg of Rhodiola rosea root extract. In an embodiment of the invention, the dietary supplement is formulated so that a daily dose provides about 50 mg, about 100 mg, about 150 mg, about 200 mg, or about 250 mg of Rhodiola rosea root extract. In a preferred embodiment, the dietary supplement is formulated so that a daily dose provides about 150 mg of Rhodiola rosea root extract.

### Ashwaghanda

Ashwaghanda is an all-natural, stress-reducing, cognitive and mood-enhancing dietary supplement derived from ashwagandha root extract. It contains citicoline, which has been shown to restore brain energy by raising levels of important neurotransmitters, increasing overall focus, and mental performance. It also promotes neural communication by delivering brain healthy polyphenols that are effective at enabling neurons to communicate more effectively, leading to a quicker and sharper mind.

Other health benefits associated with ashwaghanda are supporting healthy cortisol levels to significantly improve occasional stress, irritability, decrease fatigue and sleeplessness, and increase concentration and memory.

In an embodiment of the invention, the ashwaghanda is provided as ashwagandha root extract. In a preferred embodiment, the ashwagandha root extract contains about 2.5% withanolides.

In an embodiment of the invention, the dietary supplement is formulated so that a daily dose provides about 50 mg to about 200 mg of ashwaghanda. In an embodiment of the invention, the dietary supplement is formulated so that a daily dose provides about 50 mg, about 100 mg, about 150 mg, or about 200 mg of ashwaghanda. In a preferred embodiment, the dietary supplement is formulated so that a daily dose provides about 150 mg of ashwaghanda.

### Vitamins

In exemplary embodiments, the invention also includes one or more vitamins. In an exemplary embodiment, the invention includes B vitamins. In an exemplary embodiment, the invention includes vitamin B6 (for example as pyridoxine HCl) and vitamin B12 (for example as methylcobalamin) either alone of in combination.

In an embodiment of the invention, the dietary supplement is formulated so that a daily dose provides about 5 mg to about 15 mg of vitamin B6. In an embodiment of the invention, the dietary supplement is formulated so that a daily dose provides about 5 mg, about 10 mg, or about 15 mg of vitamin B6. In a preferred embodiment, the dietary supplement is formulated so that a daily dose provides about 10 mg of vitamin B6.

In an embodiment of the invention, the dietary supplement is formulated so that a daily dose provides about 400 µg to about 600 µg of vitamin B12. In an embodiment of the invention, the dietary supplement is formulated so that a daily dose provides about 400 µg, about 450 µg, about 500 µg, about 550 µg, or about 600 µg of vitamin B12. In a preferred embodiment, the dietary supplement is formulated so that a daily dose provides about 500 µg of vitamin B12.

### Excipients

In any embodiment of the invention, the dietary supplement can include additional excipients routinely used in the formulation of dietary supplements. These include, but are not limited to, carbohydrates (such as sugars), minerals, preservatives, flow agents, anti-caking agents, and natural flavors.

In an embodiment of the invention, the dietary supplement contains carbohydrates, including sugars, citric acid, natural flavors, silica and highly refined steviol glycosides.

In embodiments, excipient ingredients are of natural origin or naturally derived, rather than being synthetic, i.e., created entirely in a lab.

### Uses

According to the invention, the dietary supplement is added to a liquid, shaken and/or stirred until mixed well, and consumed or administered by drinking. The resulting alkaline solution/suspension is then administered orally. The combination of ingredients in an alkaline liquid provides superior synergistic effects with improved activity as compared to the individual effects of the components. The liquid can be, for example, water, coffee, tea, fruit or vegetable juice, or a powdered drink mix reconstituted in water. Water is a particularly preferred liquid because it avoids additional components that could adversely affect the alkalinity imparted by the alkalizing agent in the dietary supplement. In exemplary embodiments, the amount of supplement and water provides a pH of from about 8 to about 11, as described above.

According to an exemplary embodiment of the invention, an effective amount of the dietary supplement is administered or consumed once a day. According to this embodiment, the effective amount is added to about 8-10 ounces (235-300 mL) of a liquid, preferably water, and then shaken and/or stirred until mixed well. The resulting alkaline solution/suspension is then administered orally. The combination of ingredients in an alkaline water provides superior synergistic effects with improved activity as compared to the individual effects of the components.

According to the invention, a method of supporting and/or maintaining brain function by orally administering to a subject an effective amount of the dietary supplement as an alkaline beverage is provided. Brain functions that can be maintained include, but are not limited to, mental focus, clarity, long-term memory, short-term memory, analytical reasoning, higher learning, mental processing, mental clarity, creativity, procrastination, willpower, reasoning, mood, retrieval/recall, learning gradient, and reaction time. According to an embodiment, the dietary supplement of the invention is administered to a mentally healthy individual to support and maintain normal brain function that may typically decline due to aging or ailments.

According to the invention, a method of improving brain function by orally administering to a subject in need thereof an effective amount of the dietary supplement as an alkaline beverage is provided. Brain functions that can be improved include, but are not limited to, mental focus, clarity, long-term memory, short-term memory, analytical reasoning, higher learning, mental processing, mental clarity, creativity, procrastination, willpower, reasoning, mood, retrieval/recall, learning gradient, and reaction time. The dietary supplement of the invention has surprisingly been found to be particularly effective at improving short-term memory. These effects can have beneficial impact on handling symptoms of ADHD. It is also possible the supplement could potentially have beneficial impact on the libido.

According to the invention, a method of treating or preventing cognitive dysfunction in a subject in need thereof by orally administering an effective amount of the dietary supplement as an alkaline beverage is provided. In embodiments of the invention, the cognitive dysfunction is dementia or AD.

According to an embodiment of the invention, an effective amount of approximately 9.5 grams of an exemplary formulation includes

| | |
|---|---|
| 1 g | Total Carbohydrate |
| <1 g | Total Sugars |
| 10 mg | Vitamin B6 (as pyridoxine HCl) |
| 500 µg | Vitamin B12 (as methylcobalamin) |
| 50 mg | Magnesium (as magnesium carbonate) |
| 105 mg | Sodium (as sodium bicarbonate) |
| 430 mg | Potassium (as potassium bicarbonate and potassium carbonate) |
| 100 mg | Coffee berry whole fruit powder |
| 50 mg | Natural caffeine from Coffea robusta |
| 1000 mg | Acetyl-L-Carnitine |
| 200 µg | Huperzine-A (from Huperzia serrata whole herb extract) |
| 200 mg | L-Theanine |
| 1000 mg | Beet root extract |
| 600 mg | Alphasize™ Alpha GPC 50% |
| 200 mg | Lion’s mane mushroom |
| 300 mg | Bacopa extract |
| 150 mg | Rhodiola rosea root extract (3% rosavins, 1% salidrosides) |
| 150 mg | Ashwagandha root extract (2.5% withanolides) |
| 600 mg | SerinAid^{™} Phosphatidylserine 50% |

The effective amount, which may be administered or consumed in a single serving per day, or split into multiple servings, is sufficient to provide the beneficial effects of the invention. In exemplary embodiments, the supplement is consumed in a single serving. According to an embodiment of the invention, the dietary supplement is taken within the first hour of waking. For example, in an embodiment of the invention, the dietary supplement is taken within the first hour of waking and at least 30 minutes before eating.

According to an embodiment of the invention, within about 1 hour of administration of the dietary supplement blood flow to the brain increases, giving the subject a feeling of calm energy. According to an embodiment of the invention, within about 1-6 hours of administration of the dietary supplement energy increases, neurotransmitters activate and reduce anxiety, and memory and focus are enhanced. According to the invention, within about 6-8 hours of administration of the dietary supplement there is an increase in brain-derived neurotrophic factor (BDNF) which activates enzymes to promote gut health, digestion, and fights off midday slump. According to the invention, these effects are sustained through hours 8-12 post-administration and maintains calm energy through the end of the day.

According to the invention, the dietary supplement delivers its immediate effects (described further below) within the first week of daily administration. According to the invention, the dietary supplement delivers its long-term effects (described further below) after continued administration. Accordingly, treatment can continue for about one week, about two weeks, about three weeks, about four weeks, about five weeks, about six weeks, about seven weeks, about eight weeks, about nine weeks, about 10 weeks, about 11 weeks, about 12 weeks or indefinitely, until the desired endpoint is reached. Long term effects generally continue to improve up to and after about 84 days, or about 12 weeks, of daily administration. However, the long-term effects may be achieved sooner, and may continue to improve for some time beyond the 84 days as daily administration continues. In order to maintain the effects, the supplement should be regularly consumed on a daily basis after the desired endpoint is reached.

The dietary supplement according to the invention delivers a plethora of immediate and long-term cognitive enhancing effects.

### Immediate Effects

Immediate effects of the dietary supplement of the invention are attributed primarily to Alpha-GPC, and can be further advanced by the inclusion of Bacopa, coffee fruit concentrate, L-theanine, beet root extract, ashwaghanda, and acetyl-L-carnitine. These immediate effects can include, but are not limited to, the following:
i. Boosting of mental energy.
ii. Increased focus and concentration.
iii. Increased attention.
iv. Improved in clarity, such as mental clarity.
v. Better mood.
vi. Quicker reaction time.
vii. Sounder analytical thinking.
viii. Better reasoning skills.
ix. Enhanced flow state (i.e. the feeling of being “in the zone”).
x. Clearing the “brain” fog (i.e. mental clarity).

### Long-Term Effects

Long-term effects are achieved after prolonged use. In an embodiment, prolonged use can be at least 30 days, at least 45 days, at least 60 days, or at least 84 days of daily administration of the dietary supplement of the invention. In an embodiment, prolonged use can be at least four weeks, at least eight weeks, or at least 12 weeks of daily administration of the dietary supplement of the invention. Long-term effects of the dietary supplement of the invention are attributed primarily to PS, and can be further advanced by the inclusion of Bacopa, acetyl-L-carnitine, lion’s mane mushroom, L-theanine, Rhodiola rosea root extract, Huperzine-A, beet root extract, and ashwaghanda. These long-term effects can include, but are not limited to, the following:
i. Prevention and/or slowing of cognitive decline.
ii. Improved short-term and/or long-term memory.
iii. Increased executive functioning, such as compartmentalizing and prioritizing incoming information to the pre-frontal cortex, thereby reducing frustration; increasing the ability to handle multiple tasks simultaneously relating to retention of information and “working memory” (has beneficial outcome on symptoms associated with ADHD).
iv. Higher learning skills.
v. Better reasoning skills.
vi. Increased verbal/numerical recall.
vii. Improved mental health.
viii. Reduced stress.

According to the invention, individuals who are sensitive to caffeine should start with half a dose to assess individual tolerance.

According to the invention, the composition is not intended for children.

According to the invention, pregnant or nursing women, individuals with a medical condition, and those individuals taking medication should consult a healthcare professional before use.

The following non-limiting examples further describe aspects of the invention. These are exemplary and modifications of the precise examples can be made in view of the skill in the art and the description provided above.

### EXAMPLE 1- GENERAL INVENTIVE FORMULATION

A general formulation according to the invention includes the ingredients shown in Table 1A, below. The amounts are provided are for a daily administration in a single daily dose.

TABLE 1A

Exemplary Inventive Composition

| | |
|---|---|
| Ingredient | Amount |
| Alpha GPC | 200-400 mg |
| Phosphatidylserine | 200-400 mg |
| as Magnesium (present as magnesium carbonate) | 40-60 mg |
| Sodium (present as sodium bicarbonate) | 90-120 mg |
| Potassium (present as a combination of potassium carbonate/bicarbonate) | 300-500 mg |

Other ingredients: Preservatives, other actives, and excipients.

### EXAMPLE 1B - ADDITIONAL INGREDIENTS

Compositions according to the invention can include one or more additional ingredients Additional ingredients and exemplary amounts of each in a single daily dose are shown in Table 1B, below.

TABLE 1B

Additional Ingredients and Exemplary Amounts

| | |
|---|---|
| Coffee berry whole fruit powder | 50-100 mg |
| Natural caffeine from Coffea robusta | 20-80 mg |
| Acetyl-L-Carnitine | 500-1500 mg |
| Huperzine-A (from Huperzia serrata whole herb extract) | 100-300 µg |
| L-Theanine | 150-250 mg |
| Beet root extract | 500-1500 mg |
| Organic lion’s mane mushroom | 150-250 mg |
| Bacopa extract | 200-400 mg |
| Rhodiola rosea root extract (3% rosavins, 1% salidrosides) | 50-250 mg |
| Ashwagandha root extract (2.5% withanolides) | 50-200 mg |

### EXAMPLE 2 - EXEMPLARY INVENTIVE FORMULATION

An exemplary formulation according to the invention includes the ingredients shown in Table 2, below.

TABLE 2

Exemplary Inventive Composition

| | |
|---|---|
| Serving size: 7.9 g | Amount Per Serving |
| Calories | 15 |
| Total Carbohydrate | 1 g |
| Total Sugars | <1 g |
| Vitamin B6 (as pyridoxine HCl) | 10 mg |
| Vitamin B12 (as methylcobalamin) | 500 µg |
| Magnesium (as magnesium carbonate) | 50 mg |
| Sodium (as sodium bicarbonate) | 105 mg |
| Potassium (as potassium bicarbonate and potassium carbonate) | 430 mg |
| Coffee berry whole fruit powder | 100 mg |
| Natural caffeine from Coffea robusta | 50 mg |
| Acetyl-L-Carnitine | 1000 mg |
| Huperzine-A (from Huperzia serrata whole herb extract) | 200 µg |
| L-Theanine | 200 mg |
| Beet root extract | 1000 mg |
| Alphasize™ Alpha GPC 50% | 600 mg |
| Organic lion’s mane mushroom | 200 mg |
| Bacopa extract | 300 mg |
| Rhodiola rosea root extract (3% rosavins, 1% salidrosides) | 150 mg |
| Ashwagandha root extract (2.5% withanolides) | 150 mg |
| SerinAid™ Phosphatidylserine 50% | 600 mg |

Other ingredients: Citric acid, natural flavors, silica and highly refined steviol glycosides.

### Summary

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

1-4. (canceled)

5. The dietary supplement of claim 9, wherein the alkalizing agent provides:
a. about 50 mg of magnesium present as magnesium carbonate;
b. about 105 mg of sodium present as sodium bicarbonate; and c. about 430 mg of potassium present as a combination of potassium carbonate and potassium bicarbonate.

6. The dietary supplement according of claim 9, wherein the alkalizing agent provides about 40-60 mg of magnesium present as magnesium carbonate, about 90-120 mg of sodium present as sodium bicarbonate, and about 380-480 mg of potassium present as a combination of potassium carbonate and potassium bicarbonate in the dietary supplement.

7. The dietary supplement of any one of claim 9, further comprising one or more of vitamin B6 and vitamin B12.

8. (canceled)

9. A dietary supplement comprising about 200-400 mg of alpha-glyceryl phosphoryl choline; about 200-400 mg of phosphatidylserine; an alkalizing agent in an amount to provide about 40-60 mg of magnesium present as magnesium carbonate, about 90-120 mg of sodium present as sodium bicarbonate, and about 380-480 mg of potassium present as a combination of potassium carbonate and potassium bicarbonate in the dietary supplement; and one or more additional components selected from the group consisting of:
i. about 50-150 mg of coffee berry whole fruit powder;
ii. about 20-80 mg of caffeine;
iii. about 50-1500 mg of acetyl-L-carnitine;
iv. about 100-300 mcg of huperzine-A;
v. about 150-250 mg of L-theanine;
vi. about 500-1500 mg of beet root extract;
vii. about 150-250 mg of lion’s mane mushroom;
viii. about 200-400 mg of bacopa or Bacopa extract;
ix. about 50-250 mg of Rhodiola rosea root extract; and
x. about 50-200 mg of ashwagandha root extract.

10. The dietary supplement of claim 9, wherein the dietary supplement comprises
about 300 mg of alpha-glyceryl phosphoryl choline;
about 300 mg of phosphatidylserine;
an alkalizing agent in an amount to provide about 50 mg of magnesium present as magnesium carbonate, about 105 mg of sodium present as sodium bicarbonate, and about 430 mg of potassium present as a combination of potassium carbonate and potassium bicarbonate in the dietary supplement; and
one or more additional components selected from the group consisting of:
i. about 100 mg of coffee berry whole fruit powder;
ii. about 50 mg of caffeine;
iii. about 1000 mg of acetyl-L-carnitine;
iv. about 200 mcg of huperzine-A;
v. about 200 mg of L-theanine;
vi. about 1000 mg of beet root extract;
vii. about 200 mg of lion’s mane mushroom;
viii. about 300 mg of bacopa or Bacopa extract;
ix. about 150 mg of Rhodiola rosea root extract; and
x. about 150 mg of ashwagandha root extract.

11. The dietary supplement of claim 9, wherein the huperzine-A is derived from Huperzia serrata whole herb extract.

12. The dietary supplement of claim 9, wherein the beet root extract is oxygen enriched.

13. The dietary supplement of claim 9, wherein the Rhodiola rosea root extract comprises 3% rosavins and 1% salidrosides.

14. The dietary supplement of claim 9, wherein the ashwagandha root extract comprises 2.5% withanolides.

15. The dietary supplement of claim 9, further comprising about 10 mg of vitamin B6 and about 500 mcg of vitamin B12.

16. The dietary supplement of claim 9, wherein the supplement further comprises citric acid, natural flavors, silica, and highly refined steviol glycosides.

17. The dietary supplement of claim 9, wherein the supplement is formulated as a fine powder.

18. A method of supporting and maintaining brain function comprising orally administering an effective amount of the dietary supplement of claim 9.

19. (canceled)

20. A method of improving brain function comprising orally administering an effective amount of the dietary supplement of claim 9.

21. (canceled)

22. The method of claim 20, wherein improving brain function comprises improving at least one of mental focus, clarity, long-term memory, short-term memory, analytical reasoning, higher learning, mental processing, mental clarity, creativity, procrastination, willpower, reasoning, mood, retrieval/recall, learning gradient, and reaction time.

23. A method of treating or preventing cognitive dysfunction in subject in need thereof by orally administering an effective amount of the dietary supplement of claim 9.

24. The method of claim 23, wherein the cognitive dysfunction is dementia.

25. (canceled)

26. The method of claim 20, wherein a daily dose of said dietary supplement with a total mass of about 9.5 g is added to about 8-10 ounces of a liquid prior to administration.

27. The method of claim 26, wherein the liquid is water.
